(19) United States
(12) Patent Application Publication
(10) Pub. No.: US 2015/0296770 A1
(43) Pub. Date: Oct. 22, 2015

(54) APPARATUS FOR EXTRACORPOREAL CELLULAR THERAPY OF LUNG OR OTHER ORGAN

(71) Applicants: Mark Frings PITTENGER, Baltimore, MD (US); Pablo Gerardo SANCHEZ, Baltimore,, MD (US)

(72) Inventors: Mark Frings PITTENGER, Severna Park, MD (US); Pablo Gerardo SANCHEZ, Baltimore, MD (US)

(21) Appl. No.: 14/435,629

(22) PCT Filed: Oct. 15, 2013
(86) PCT No.: PCT/US13/65033

§ 371 Date: Apr. 14, 2015

Related U.S. Application Data
(60) Provisional application No. 61/713,832, filed on Oct. 15, 2012.

(51) Int. Cl. A01N 1/02; A61K 35/28

(52) U.S. Cl. CPC A01N 1/0247; A01N 1/0226; A61K 35/28

(57) ABSTRACT

This application describes an apparatus and a method for the treatment of a damaged lung, heart, or other organ or tissue using cells contained in the apparatus and a liquid interface that allows communication with the circulation of the damaged organ or tissue. The apparatus can be used to treat the organ inside or outside the body. The apparatus is designed to sit outside the body but a similar apparatus is envisioned that is implantable. The apparatus contains a porous material or matrix that the cells are grown on, and the volume of the apparatus provides that it can contain a therapeutically useful number of cells in the range 0.5×10e6 to 200×10e6.

### SUMMARY

This application describes an apparatus and a method for the treatment of a damaged lung, heart, or other organ or tissue using cells contained in the apparatus and a liquid interface that allows communication with the circulation of the damaged organ or tissue. The apparatus can be used to treat the organ inside or outside the body. The apparatus is designed to sit outside the body but a similar apparatus is envisioned that is implantable. The apparatus contains a porous material or matrix that the cells are grown on, and the volume of the apparatus provides that it can contain a therapeutically useful number of cells in the range 0.5×10e6 to 200×10e6. The cells are contained in the apparatus and a filter with 1 micron pores prevents cells from being transported to the organ or tissue. The apparatus is connected to the organ or tissue in a way that allows fluid flow from the organ or tissue to the apparatus and from the apparatus to the organ or tissue of interest. This may be the same organ/tissue or a different organ/tissue. The fluid flow may be re-circulated from the apparatus to the organ/tissue. The cells contained in the apparatus may produce growth factors, cytokines or other substances that are useful to inhibit inflammation, enhance tissue and organ recovery from injury or disease, and stimulate growth of new blood vessels and aid in forming new tissue. The cells in the apparatus can be modified to express additional therapeutic molecules. The cells in the apparatus can be immortalized cells that provide a consistent and continuous source of the cells that can be carefully evaluated and provide a reliable production of the desired growth factors, cytokines and other substances. The cells in the apparatus can respond to signals (molecules) from the injured or diseased organ or tissue and change their expression of cytokines growth factors and other substances. The cells in the apparatus can be immortalized cells and provide these growth factors and cytokines or the immortalized MSCs can be genetically modified to express additional factors of therapeutic value.

### BACKGROUND

Lung donor quality continues to be a major hurdle in lung transplantation, hampering the number of procedures performed every year; merely 15-20% of available multi-organ donors become lung donors. In addition, donor quality is a significant determinant of recipient survival after transplantation.

Several mechanisms define the quality of the donated lung, mostly related to the inflammatory response elicited by brain death, mechanical ventilation, gastric aspiration, trauma, and cold ischemic storage. These responses ultimately induce primary graft dysfunction and acute rejection, leading to a decrease in organ and patient survival by favoring chronic rejection.

The lung's architecture presents a complex dilemma in terms of organ preservation and reconditioning. Cells in the endothelial and epithelial surfaces have been implicated with the production of molecular markers that regulate organ dysfunction and repair. The endothelium appears to be a predominant source of oxidants, upregulated adhesion molecules, prothrombotic and antifibrinolitic factors that lead to microvascular thrombosis compromising blood flow after reperfusion. In addition, brain death and prolonged cold ischemia favor the loss of the water-containing properties in the alveolar epithelium. This leads to the development of pulmonary edema, which compromises lung oxygen exchange and cellular viability.

Recently, ex vivo perfusion system has been developed and has the ability to maintain lungs outside the body for many hours. This system, that does not require the use of blood or blood components has a proprietary buffered acellular solution, with plasma-like osmotic and oncotic pressures, and maintains the lungs for extended periods of time outside the body without adversely impacting their physiology.

We propose that a apparatus containing stem cells could provide important growth factors and cytokines during ex vivo lung perfusion to maintain or improve the lung health ex vivo. This apparatus and procedure could become an important therapeutic option for organ reconditioning before transplantation, ultimately improving the quality of the lung to be transplanted, and increasing the number of usable lungs and transplant survival.

The Ex-vivo Cellular Therapy apparatus (XCT-apparatus) can be used to treat lungs ex-vivo. The cells in the apparatus produce growth factors and cytokines that can enhance tissue repair in the lung. It can also be used to treat the lungs in situ as may be needed to treat infection or idiopathic lung disease. The cells in the XCT-apparatus can respond to signals from damaged tissue to produce different growth factors and cytokines that enhance tissue repair. A related implantable in vivo cellular therapy apparatus (ICT-apparatus) is envisioned.

In one proposed use of the XCT-apparatus, the apparatus will be connected in-line with the ex vivo perfusion system and receive the fluid from the pulmonary vein. The cells in the apparatus will be perfused by the post-lung fluid. This will bring into the apparatus a representative molecular sample of the lung's inflammatory status triggering an adaptive response from the cells in the apparatus such that the cells alter their “paracrine secretome” that is, the molecules secreted from the cells including beneficial factors (including molecules that are growth factors and cytokines). These factors will return into the lung through the perfusion solution and interact with the resident lung cells and inflammatory cells and down regulate inflammation, to favor endothelial/epithelial stability. This will cause re-establishing a better and more normal alveolar-capillary barrier ultimately improving organ functionality.

Several factors produced by MSCs could be involved in the resuscitation of these injured organs. Some of these factors secreted by MSCs that could down-regulate inflammation and modulate endothelial/epithelial dysfunction are:
1—Interleukin 1 receptor antagonist (IL-1ra). Interleukin-1 pathway plays an important role in the generation of sterile inflammation similarly to the effects of tumor necrosis factor alpha (TNFalpha) in infectious inflammation. IL-1ra secreted by MSCs blunts the effects of IL-1 and TNFalpha attenuating inflammation by reprograming macrophages from the pro-inflammatory type (M1) to the anti-inflammatory phenotype (M2). Other cytokines secreted by MSCs-IL-10, TNFalpha stimulated gene/protein 6 (TSG-6) and PGE2 may contribute to down-regulate inflammation by a similar mechanism, ultimately decreasing the amplification of pro-inflammatory signals by the lung cells.
2—Angiopoietin-1 (Ang1). Ang1 is a growth factor that stabilizes the endothelium, reduces its permeability and inhibits leukocyte-endothelium interactions by modifying endothelial cell adhesion molecules and cell junctions. Additionally, it has been reported to reduce protein permeability in alveolar epithelial type II cell cultures.
3—Keratinocyte growth factor (KGF). KGF is a growth factor that stimulates proliferation of epithelial cells. In the lung, KGF has been found to normalize fluid balance by increasing vectorial fluid transport across the alveolar epithelium in part through increased trafficking of sodium transport proteins to the cell surface.

List of factors produced by MSCs that inhibit inflammation: TGFbeta, HGF, PGE2, Gal-1, iNOS, IL-6, CD73, IL-1Rag, IL-10, HLA-G, IDO, TSG-6

List of growth factors produced by MSCs: M-CSF, G-CSF, GM-CSF, LIF, SCF, Flt-3 Ligand, TPO, SDF-1

As opposed to injecting the therapeutic cells into the body, the advantages of having the cells on an extracorporeal apparatus with a downstream inline filter are many. The cells contained on the apparatus do not enter the body so there are many fewer biohazard concerns - such as the risk of embolism due to cell aggregation and blood vessel clogging. There is no risk of introduction of immortalized or cancerous cells into the body. The quality and viability of the cells in the apparatus can be checked over time, and replaced with a new apparatus if deemed necessary. The apparatus can be disconnected from the organ or patient if it is not having the desired effect and other treatments initiated.

What is claimed is:

1. An apparatus with an inlet and outlet and containing cells and these resident cells can respond to input chemicals, biochemicals and/or one or more additional cell types to produce a useful or beneficial substance(s) from the apparatus resident cells.

2. An apparatus with an inlet and outlet and containing immortalized resident cells and the resident cells can respond to input chemicals, biochemicals and or one or more additional cell types to produce a useful or beneficial substance(s) from the cells.

3. An apparatus of claim 1 or claim 2 is connected via catheters to deliver a beneficial effect to the lung ex vivo prior to transplantation.

4. An apparatus of claim 1 or claim 2 wherein it is connected via catheters to the lung in vivo.

5. An apparatus of claim 1 with an inlet and outlet that contains resident cells and can be connected to a patient's circulatory system to deliver a therapeutic effect for the purpose of treating lung insufficiency or injury.

6. An apparatus of claim 1 where the resident cells are stem cells.

7. The apparatus of claim 1 where the resident cells are mesenchymal stem cells.

8. The apparatus of claim 2 where the resident cells are immortalized stem cells.

9. The apparatus of claim 2 where the resident cells are immortalized mesenchymal stem cells.

10. An apparatus of claim 3 wherein the apparatus is connected immediately proximal to the main blood supply of the organ.

11. An apparatus of claim 3 wherein the apparatus is connected immediately proximal to the main blood supply of the organ AND receives partial flow from the immediately distal blood flow from the organ.

12. A process where the apparatus in claim 1 or claim 2 is connected to a patient's organ of interest to deliver a beneficial therapeutic effect in vivo.

13. A process where the apparatus in claim 1 or claim 2 is connected to an organ of interest to deliver a beneficial effect ex vivo.

14. An apparatus with an inlet and outlet that contains resident cells and can be connected to an organ to deliver a therapeutic effect to the organ ex vivo.

15. An apparatus with an inlet and outlet that contains resident cells and can be connected to an organ to deliver a therapeutic effect to the organ in vivo.
